(19) United States
(12) Patent Application Publication
(10) Pub. No.: US 2006/0025796 A1
(43) Pub. Date: Feb. 2, 2006

(54) Methods and apparatus for minimally invasive recovery of bone and/or other tissue

(75) Inventor: Orlando Merced-O'Neill, Archer, FL (US)

Correspondence Address: MCANDREWS HELD & MALLOY, LTD, 500 WEST MADISON STREET, SUITE 3400, CHICAGO, IL, 60661, US

(73) Assignee: Regeneration Technologies

(21) Appl. No.: 10/903,417

(22) Filed: Jul. 30, 2004

(51) Int. Cl. A61B 17/00

(52) U.S. Cl. 606/190

(57) ABSTRACT

Methods and apparatus are provided for minimally invasive recovery of bone and other tissues. The methods include separating bone or other tissue from surrounding tissue in a minimally invasive fashion. The methods may be used for recovering an essentially intact bone from a donor with reduced detriment to the aesthetic appearance of the donor. The instruments include an elongated shaft and a tissue separator attached to one of the ends of the shaft. The separator may have a moderately sharp edge and/or a shape that is reciprocal to at least a portion of the shape of a bone. Kits are provided comprises an elongated shaft and two or more tissue separators which are attachable to the shaft. The separators may have substantially similar shapes but have different sizes, and/or may be different shapes.

### FIELD OF THE INVENTION

Methods and apparatus are provided for minimally invasive recovery of bone and other tissues. The methods include separating bone or other tissue from surrounding tissue in a minimally invasive fashion. The methods may be used for recovering an essentially intact bone from a donor with reduced detriment to the aesthetic appearance of the donor. The instruments include an elongated shaft and a tissue separator attached to one of the ends of the shaft. The separator may have a moderately sharp edge and/or a shape that is reciprocal to at least a portion of the shape of a bone. Kits are provided comprises an elongated shaft and two or more tissue separators which are attachable to the shaft. The separators may have substantially similar shapes but have different sizes, and/or may be different shapes.

### BACKGROUND OF THE INVENTION

Minimally invasive surgical techniques are aimed at reducing the amount of surrounding tissue which is damaged during diagnostic or surgical procedures, thereby reducing patient recovery time, discomfort, and deleterious side effects. For surgery on a living patient, it is readily apparent that use of minimally invasive techniques are desirable. However, for recovery of bone for transplant from a donor, it is not so apparent that there is still a benefit from employing minimally invasive techniques.

Conventional methods of bone and tissue procurement use incisions that travel the length of the donor limb. Bohatyrewicz et al., “Regional program for bone allograft harvesting following multiorgan procurement in Pomeranian Academy of Medicine in Szczecin”, Cell and Tissue Banking, 3:55-58 (2002) shows the effects of conventional methods of bone procurement using incisions along the length of the donor limb. Bohatyrewicz also describes difficulty returning the donor to acceptable aesthetics for funeral arrangements.

U.S. Pat. No. 5,522,827 (Zimmer, Inc.) discusses an apparatus and method for harvesting a tendon graft beneath the overlying tissue through a remote incision which exposes only a portion of the donor tendon from which the graft will be harvested. The apparatus includes an elongated tubular shaft connected to a cylindrical handle and a length of flexible cutting wire or cable. The method of using the apparatus includes making an incision to expose only a portion of said donor tendon at the proximal end of the tendon graft to be harvested; slitting or stripping said donor tendon longitudinally to form the tendon graft by passing the cable loop laterally through or around the donor tendon and the moving the cable loop along the donor tendon beneath the overlying tissue; and severing said tendon graft at its distal end beneath the overlying tissue by closing the cable loop.

U.S. Patent Application Publication No. U.S. 2004/0059338 A1 (Maxilon Laboratories, Inc.) discusses a bone grafting and/or shaping instrument that includes a handle portion, a collection chamber and a blade. Features on the blade cooperate with features on the collection chamber to secure the blade to the collection chamber. A flexible joint between the collection chamber and the handle portion allows the user to orient the cutting edge of the blade in any desired position relative to the handle portion.

International Publication No. WO 03/002022 (Depuy Products, Inc.) discusses apparatus and methods for use in the performance of minimally invasive orthopaedic procedures. Such procedures include a minimally invasive autologous bone graft harvesting procedure. An instrument such as a trocar is inserted through a small stab incision in the skin of the patient, and corticocancellous strips or other portions of the ilium are removed.

### SUMMARY OF THE INVENTION

As one aspect of the invention, methods are provided for separating a bone from surrounding tissue with reduced detriment to the surrounding tissue. The methods comprise (a) making an incision which provides access to the bone, (b) positioning a moderately sharp edge at a surface of the bone through the incision, and (c) moving the moderately sharp edge between the bone and the surrounding tissue for at least part of the length of the bone. By positioning and moving the moderately sharp edge in this fashion, the moderately sharp edge separates surrounding tissue from the bone with reduced or minimal damage to the surrounding tissue. Preferably, the incision is a relatively small incision and/or is located at approximately at an end of the bone. Most preferably, the incision is a strategic incision, as described herein.

As another aspect of the present invention, minimally invasive methods are provided for separating a bone from soft tissue surrounding the bone in a living subject. The methods comprise (a) making an incision which provides access to the bone, (b) positioning a moderately sharp edge at a surface of the bone through the incision, (c) pushing the moderately sharp edge between the bone and the surrounding tissue so that the soft tissue is separated from the bone, leaving the surrounding soft tissue essentially intact. The soft tissue may comprise a vascular network, and step (c) can include separating the soft tissue from the bone without destroying the vascular network of the separated soft tissue.

As yet another aspect of the present invention, methods of separating soft tissue from bone in a living patient are provided. The methods minimize scar tissue and trauma to the patient. The methods comprise (a) making an incision within the tissue at or near the proximal or distal third of the bone; (b) accessing the bone through the incision with an instrument comprising a moderately sharp edge; (c) moving the moderately sharp edge along the bone to separate at least some of the surrounding soft tissue from the bone.

As a further aspect of the present invention, methods are provided for recovering essentially intact bones or a substantial portion of a bone from a donor with reduced detriment to the appearance of the donor. The methods comprise (a) separating the bone from essentially all surrounding soft tissue; (b) removing the bone (for example, an essentially intact bone, or a substantial portion of the bone) from the donor's body; and (c) leaving the soft tissue essentially intact in the donor's body.

As another aspect of the present invention, methods are provided for separating a bone from soft tissue surrounding the bone in a minimally invasive fashion. The methods comprise making an incision within the distal or proximal third of the bone and separating the surrounding tissue from at least a portion of the length of the bone. The incision is made approximately at one end of the bone, and the bone and the tissue are accessed through the incision.

As another aspect of the present invention, methods are provided for recovering an essentially intact bone or a substantial portion of a bone from a donor with reduced detriment to the aesthetic appearance of the donor. The donor may be a living patient or a cadaver. The methods comprise making an incision approximately at one end of the bone, separating soft tissue surrounding the bone along at least a portion of the length of the bone by accessing the bone and the tissue through the incision, and removing the essentially intact bone or a substantial portion of the bone through the incision. As yet another aspect of the present invention, methods of recovering tendon grafts are provided. The methods comprise separating the tendon from an associated muscle; removing the tendon; and leaving most of the associated muscle essentially intact in the donor's body. The term “tendon” as used herein is intended in its broad sense and refers to fibrous connective tissue for use in grafts, such as, but not limited to, tendons, ligaments and demineralized bone.

As another aspect of the present invention, instruments are provided for separating a bone from soft tissue surrounding the bone in a minimally invasive fashion. The instruments comprise an elongated shaft having a length and two ends, and a tissue separator attached to one of the ends of the shaft. The tissue separator having a moderately sharp edge, the edge being essentially perpendicular to the length of the elongated shaft. Alternatively, the edge may be oriented at an acute or oblique angle with respect to the shaft. The edge may have either a leading offset or a trailing offset with respect the shaft.

As another aspect of the present invention, instruments are provided for separating two soft tissues from one another in a minimally invasive fashion. The instruments comprise an elongated shaft having a length and two ends, and a rigid tissue separator attached to one of the ends of the shaft. The tissue separator has a moderately sharp edge, and the edge is essentially perpendicular to the length of the elongated shaft.

As another aspect of the present invention, instruments are provided for separating an Achilles tendon from a donor in a minimally invasive fashion. The instrument comprises an elongated shaft having a length and two ends, and a tissue separator attached at one end of the shaft, wherein the separator comprises a scoop.

As yet another aspect of the present invention, kits are provided for procuring an essentially intact bone or a substantial portion of a bone from a donor with reduced detriment to the aesthetic appearance of the donor. The kits comprise an elongated shaft having a length and two ends and two or more tissue separators. The tissue separators are selected from the group consisting of: separators comprising a separator edge having a semi-circular shape; separators comprising a separator edge having a circular shape; separators comprising a separator edge having a J-shape; separators comprising a separator edge having a C-shape; separators comprising a separator edge having a U-shape; and separators comprising a separator edge having a scoop shape. Alternatively, tissue separators may comprise other shapes or combinations of these and other shapes.

### BRIEF DESCRIPTION OF SEVERAL VIEWS OF THE DRAWINGS

FIG. 1 shows a shaft and handle suitable for an instrument for minimally invasive bone recovery.

FIG. 2 shows a separator that is well suited for separating a femur from surrounding tissue.

FIG. 3 shows a separator that is well suited for separating a tibia from surrounding tissue.

FIG. 4 shows a separator that is well suited for separating a fibula from surrounding tissue.

FIG. 5 shows a separator that is well suited for separating a tendon or a portion of a tendon from surrounding tissue.

FIG. 6 shows a separator that is well suited for separating an ilium, or a rib, or a mandible, or a scapula from surrounding tissue.

FIGS. 7(a) and 7(b) show separators that are well suited for separating an Achilles tendon from surrounding tissue.

FIG. 8 shows a separator attached to a shaft with a cam arrangement.

FIG. 9 shows a separator attached to a shaft with a bolt.

FIG. 10 shows a separator attached to a shaft with a leaf spring arrangement.

FIG. 11 shows a separator attached to a shaft with a cut-out configuration.

FIG. 12 shows a separator attached to a shaft with a T-shaped attachment.

FIG. 13 shows an exploded view of one embodiment of an instrument for separating bone from surrounding tissue.

FIG. 14 shows the use of one embodiment of an instrument for separating bone from surrounding tissue.

FIG. 15 shows a sternal knife well suited for opening a donor's chest cavity.

FIG. 16 shows a knife well suited for separating skin or fascia.

FIG. 17 shows a curved knife well suited for dissecting and/or transecting tendon, ligament, retinaculum, and/or other soft tissue.

### DETAILED DESCRIPTION OF THE INVENTION

Methods and apparatus are provided for recovering a bone and/or other tissue from a donor using minimally invasive techniques. The donor may be a human being or other mammal, and the bone or other tissue may be recovered for use as an autograft, allograft, or xenograft transplant. The donor may be a living patient for example, where the bone or other tissue is recovered for autograft transplant. Alternatively, the donor may be a deceased person who chose to donate tissue to others, which is generally the situation when an essentially intact bone or a substantial portion of a bone is to be recovered. The present minimally invasive techniques greatly facilitate the procurement of relatively large sections of bone or other tissue, including tendons, ligaments, bones, long bones, essentially intact bones, and substantial portions of bones, with reduced detriment as compared to conventional techniques. For example, the present methods and apparatus permit recovery of bone or other tissue using smaller incisions than have conventionally been used, thereby providing reduced detriment to aesthetic appearance and, in the case of a living patient, reductions in patient recovery time, discomfort, and/or deleterious side effects.

When bone is being recovered for transplant purposes, it is generally desirable to obtain an essentially intact bone, but in some situations, it may be desirable to cut the bone into substantial portions. For example, a bone may be cut into one, two or more substantial portions, so long as the substantial portions may be recombined into a recognizable form of the original bone. For example, a bone might be divided into about twelve substantial portions yet still be put back together in recognizable form.

A substantial portion of a tissue, or a transplantable tissue, such as a bone or a tendon, also includes or refers to a portion of tissue which is of sufficient size and shape to be processed by methods known in the art to produce one or more structural graft components of either a monolithic or assembled configuration. For example, a 2 cm long section cut across the diaphysis of a fibula could be processed to produce a monolithic cervical fusion graft. As another example, a 3 cm long section cut across the either the diaphysis, metaphysis or epiphysis of a femur could be processed to produce several bone planks or bone pins which could then be assembled together by methods known in the art to produce an assembled lumbar fusion graft. A substantial portion of bone would include bone pieces of sufficient size and shape that they are capable of being reconstituted as the original bone, or being processed to produce structural graft components. For example, a substantial portion of bone may be created by the breaking of a bone by traumatic injury or by the cutting of a bone before, during or after the recovery procedure.

Surrounding tissue typically include muscle, ligaments, tendons, blood vessels, and other soft tissues that adhere to bone in a typical body. The surrounding tissue may also include skin, fascia, dermis, pericardium, cartilage, nerves, organs, or connective tissues. Muscle makes up most of the surrounding tissue in terms of volume and structural significance, so it is particularly advantageous to reduce detriment to muscle when removing a bone when it is desired to maintain the aesthetic appearance of the donor. It may be desirable to remove a bone from a limb without completely cutting any muscle across its direction of motion or without cutting any muscle in a way that noticeably alters the aesthetic appearance of the donor. Surrounding tissue is essentially intact inside the donor's body when it retains one or more of the functional attributes of support, structure, shape, attachment, access, delivery or appearance. For example, a muscle mass which maintains the external appearance or shape of a limb or other body part is essentially intact in the donor's body. As another example, blood vessels which maintain the ability to deliver fluids, including embalming fluids, at a level sufficient for a given function, such as embalming of a deceased donor's body, are essentially intact. As another example, connective tissue which retains sufficient structure and strength to hold other tissues together, in place, or in shape, is essentially intact.

In some embodiments, the minimally invasive recovery techniques will generally involve strategic incisions. A strategic incision is an incision generally located and sized to allow access to the bone or other tissue to be recovered, while reducing or minimizing detriment to and disturbance of other surrounding tissue. For example, it has been found that a bone can be recovered using incisions strategically placed at points along the donor limb, thereby avoiding the need for incisions as long as the donated bone itself. A strategic incision may be made through the skin, connective tissue, muscle tissue, and/or other soft tissues. The length of a strategic incision or any incision or other opening used to access, manipulate, separate, or recover tissue will depend on the location and purpose of the incision, but may be readily determined by the recovery agent based on the teachings of the present disclosure.

In the present minimally invasive recovery techniques, the length of an incision or opening is the length of cut employed to produce the incision or opening, for example, the length of the cut generated with a scalpel. For a linear incision or opening, this is simply the length of the cut. The length for a circular incision or opening is the circumference. The length for a polygonal incision or opening is the sum of the length of the sides. The length for other geometries may be easily measured or calculated in a similar fashion. In some cases the incision may be nearly the same length as the bone to be recovered, alternatively the incision may be about ⅔ the length of the bone, alternatively the incision may be about ½ the length of the bone, alternatively the incision may be about ¼ the length of the bone, alternatively the incision may be about ⅕ the length of the bone, alternatively the incision may be about 1/10 the length of the bone.

In some embodiments of the present methods, an incision may be made having a length slightly less than the length of a common Watson-Jones type incision known in the art for recovery of tissue from the leg. Preferably, the incision has a length of less than about 50 cm, alternatively less than about 40 cm, alternatively less than about 30 cm, alternatively less than about 20 cm, alternatively less than about 10 cm, alternatively less than about 5 cm, alternatively less than about 2 cm. The size of an incision is chosen to allow access while minimizing detriment to the surrounding tissue. For example, an incision near the wrist could be sized at about 5 cm, alternatively about 15 cm, alternatively about 10 cm, alternatively about 20 cm. An incision near the shoulder or upper arm could be sized about 30 cm, alternatively about 20 cm, alternatively about 35 cm. An incision near the hip or femur could be sized about 50 cm, alternatively about 40 cm, alternatively about 55 cm.

An incision may be located within near the proximal third or distal third of the bone. As used herein, when it is said that an incision is made “within” an area of the bone, the incision is made to tissues such as skin, muscle, and/or other soft tissues at or near that area of the bone. For example, when an incision is made within the proximal third of the bone, the incision is made to tissues such as skin, muscle, and/or other soft tissues at or near that proximal third of the bone. The incision may cross over and extend past the end of the bone and into the joint area or articulation between the bone and adjacent bones. The articulation between adjacent bones includes the area around the joint. An incision which is within the proximal or distal third of a bone will not pass over into the central third of the bone. Alternatively, the incision may be made outside the central fourth of the bone. Alternatively, the incision may be made without crossing over the mid-plane of the bone or the center of the length of the bone. Alternatively, the incision may be located at the end of the bone or near the end of the bone. Alternatively, the incision may be located within an articulation between two bones or overlapping the end of one or more bones. Alternatively, the incision may be located within the central third or central portion of the bone. These incisions are large enough for the recovery agent to access the bone, for example, to manipulate tissue, to insert devices that strip or separate surrounding tissue, including soft tissue and muscle mass, away from the recovered bone and tissue. Once proper separation has occurred, these incisions are also the removal path for the procured bone and tissue, leaving the remaining muscle mass and tissue in the donor. The minimally invasive technique also reduces the required suturing after recovery, and more importantly, minimizes the aesthetic detriment of the recovery process.

The foremost challenge with minimally invasive recovery using strategic incisions is efficiently separating the bone and optionally other tissue from the muscle mass and tissue that will remain in the donor. With the larger incisions, the recovery agent can easily see the targeted tissue and bones, as well as the adjacent tissue, thereby making the recovery process simple. Minimally invasive recovery requires the recovery agent to dexterously perform the required separation with less visual information. While the performance of minimally invasive recovery may be more difficult than conventional techniques, the benefits of reduced detriment are substantial, and the present techniques may significantly reduce the difficulty associated with minimally invasive recovery. Detriment includes any negative effect or impact upon the bone or donor, such as impairment of function, feeling, or appearance.

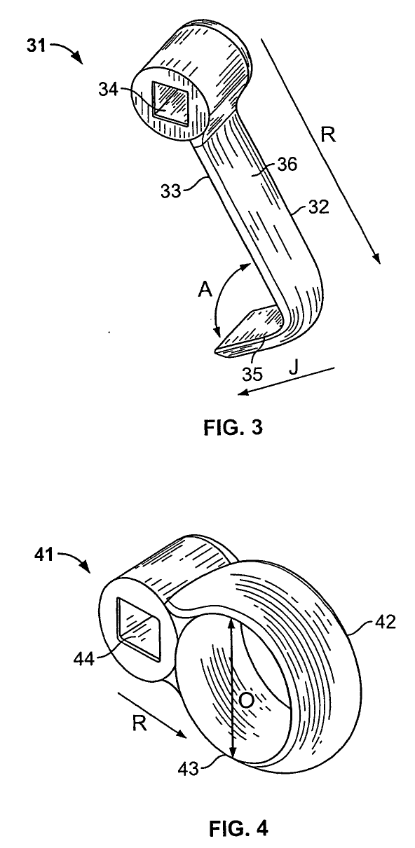

The present minimally invasive recovery techniques may be used to separate a bone from surrounding tissue with reduced detriment to the surrounding tissue. First, an incision is made which provides access to the bone. Generally, the incision will be relatively small, as compared to incisions made using conventional bone recovery techniques, which typically employ incisions that are as long as, or longer than the length of the bone to be recovered. In contrast, it has been found that much smaller incisions may be employed which still permit access to the bone. Access is provided to the bone when the incision is large enough to accommodate at least one dimension of an instrument to be used. For example, when the C-shaped separator of FIG. 2 is employed, the incision need not be as long as the entire separator, but rather may be just large enough to accommodate the width of the small edge, so that it may be maneuvered into the incision. For access to be provided, the skin of the donor must be incised, and preferably muscle between the bone and the skin is also incised.

Some type of separator is then positioned between the bone and surrounding tissue. The separator is generally attached so that its separation edge is at a right angle to the shaft, so that the force generated by back and forth movement of the shaft is applied to the intersection of bone and surrounding tissue. In some preferred embodiments, the separator includes a moderately sharp edge, and this moderately sharp edge is positioned at a surface of the bone, between the bone and the surrounding tissue. The separator is inserted into the donor through the incision, so the incision should be large enough to accommodate at least one dimension of the separator.

Preferably, the separator is a rigid tissue separator attached to one of the ends of the shaft. Alternatively, tissue separators may comprise a wire, cable, band, cord, line, strap, or flexible saw blade. Alternatively, tissue separators may be made of flexible, directionally flexible, formable, malleable, or adjustable materials such as flexible or semirigid metals, composites, or polymers which may be shaped or adjusted by hand or with some additional fixture or apparatus. Alternatively, tissue separators may exhibit sufficient compliance in a given direction or at one or more points along their length so as to self adjust or flex to accommodate the geometry of the body. Alternatively, tissue separators may use some mechanism other than simple mechanical cutting to separate tissues. Alternatively the separator may include a liquid jet or stream, a pressure field, an ultrasonic actuator, a thermal element at either high or low temperature, an electromagnetic element, a motor, or a drive mechanism.

Each of the foregoing separators (or other embodiment of a separator) is attachable to one of the ends of the shaft. The shaft may be adjustable or variable in length, and the shaft may comprise multiple shaft portions of similar or different lengths which can be assembled together to form a shaft of a desired length. Alternatively, the apparatus may further comprise a handle attached to the shaft.

After the separator is in proper position, the recovery agent then moves the separator between the bone and the surrounding tissue for at least part of the length of the bone. By positioning and moving the separator in this fashion, the surrounding tissue is separated from the bone with reduced or minimal damage to the surrounding tissue. In particular, when the separator comprises a moderately sharp edge that is used to separate the bone from the surrounding tissue, damage to the surrounding tissue is far less likely to occur than if a sharp edge were used. The separator may be pushed or pulled along the bone, or a combination of pushing and pulling may be used so as to separate surrounding tissue from a portion of the length of the bone. When the bone is to be recovered, surrounding tissue will be separated along the entire length of the bone, or at least along a majority or a substantial portion of the length of the bone. The length of the bone generally refers to its longest dimension. Long bones such as the femur have a length that is much greater than its circumference or width.

The use of a separator having a moderately sharp edge may be preferred when it is desirable to preserve, or at least reduce detriment to, the vascular network in the surrounding tissue. For example, wherein the surrounding tissue comprises a vascular network, it may be desirable to separate the surrounding tissue from the bone while leaving the vascular network of the surrounding tissue essentially intact. The use of a moderately sharp edge rather than a sharp edge to effect the separation may facilitate leaving the vascular network essentially intact. By preserving the vascular network, or at least leaving it essentially intact, one may preserve the option of passing a fluid, such as an embalming fluid, through the vascular network of the surrounding tissue. This may be desirable or even required where the donor is being prepared for burial in certain states. Funeral directors typically use embalming fluids such as formaldehyde and formaldehyde based fluids including alcohols, propylene glycol, amphyl, formalin, and phenol to preserve the decedent. Embalming fluids may also comprise powder or cream formulations of these or other components, as is known in the art.

In the present minimally invasive recovery techniques, the bone to be recovered generally has a length and two ends, and an incision is made approximately at one end of the bone, typically within about the proximal or distal third of the bone. The incision may be made through the skin, connective tissue, muscle tissue, and/or other soft tissues. In contrast to conventional procurement techniques, the present method does not require an incision such as a Watson-Jones incision or modification thereof, which runs along the entire length of the bone or along the length of more than one bone. The surrounding tissue is separated from the bone along at least a portion of the length of the bone by accessing the bone and the tissue through the incision. Alternatively, the present techniques may be employed for the separation of tissue from bone in a minimally invasive fashion even though a standard Watson-Jones type incision is used to open the skin.

Methods are also provided for recovering an essentially intact bone from a donor with reduced detriment to the aesthetic appearance of the donor. An essentially intact bone is distinguished from bone shavings, bone cores, and other relatively small pieces of bone. By way of comparison, bone shavings and bone cores do not constitute essentially intact bone or a substantial portion of bone. In some embodiments of the present invention, an incision is made across the length of the bone and approximately at one end of the bone. Soft tissue surrounding the bone is separated along at least a portion of the length of the bone by accessing the bone and the tissue through the incision. The essentially intact bone is removed through the incision. Preferably, substantially all of the non-targeted tissue is left in the donor.

The location and size of the incision(s) made in the donor are suitable for accessing and removing the bone as described herein. The incision will usually be made near the end of the bone. For example, the incision can be made at the articulation of two connected bones or near a joint, and the midpoint of the incision can be at or near the center of the articulation. Alternatively, the incision of less than ½ the length of the bone may be made at any point on the skin which provides suitable access to the bone.

Optionally a second incision can be made at the other end of the bone that is being procured, so that soft tissue along at least a second portion of the length of the bone can be separated from the bone through the second incision. Where the bone is disposed in an extremity (including but not limited to bones such as femora, tibiae, fibulae, humeri, radii, ulnae, ribs, mandibles, or scapulae), the method preferably comprises making no more than one, alternatively no more than two, alternatively no more than three incisions in that extremity. It is desirable that the incision be located no more than about 30 cm from the end of the bone. Alternatively, the incision can be made approximately at the level of the superior margin of the greater trochanter of the hip and about 2 cm above the tissue at or near the anterior portion of the iliac crest.

The location and size of the incision(s) depend in part on what bone is to be recovered and in part on the size of the donor. For example, when recovering bone from the lower extremity, the minimally invasive recovery technique can use strategic incisions at the top of the hip, the knee, and/or the ankle. The first strategic incision may start 2 cm above the posterior half of iliac crest that follows the sartorial tract, then medially or along the midline to the level of the greater trochanter of the femur, and is approximately 25 cm in length. The strategic midline incision at the knee is approximately 20 cm in length with the patella being the midpoint. A strategic incision at the ankle is approximately 15 cm in length with anterior ankle being the midpoint of the incision. As another example, when recovering bone from the upper extremity, the minimally invasive recovery methods preferably include strategic incisions at the shoulder, elbow and/or wrist, rather than a midline or posterior or anterior or lateral or medial incision running the entire length of the arm. Preferably the strategic incision at the shoulder is approximately 15 cm, and starts at the acromion process and proceeds to the level of the lesser tubercle, and the second strategic incision is approximately 10 cm with the elbow at the midpoint. Preferably, the strategic incision along the forearm is approximately 4 cm with the wrist at the endpoint or midpoint of the incision.

More generally, the strategic incisions are approximately at the joints connected to the bone that is being procured. For example, in recovering a femur from a donor, it is desirable to make the incisions approximately at a hip and a knee of the donor. In recovering a tibia from a donor, it is desirable to make the incisions approximately at a knee and an ankle. In recovering a fibula from a donor, it is desirable to make the incisions are approximately at a knee and an ankle. In recovering a humerus, it is desirable to make the incisions approximately at a shoulder and an elbow. In recovering a radius, it is desirable to make the incisions approximately at an elbow and a wrist. In recovering an ulna, it is desirable to make the incisions approximately at an elbow and a wrist. In recovering a scapula, it is desirable to make an incision approximately at a shoulder. In recovering a rib, it is desirable to make incisions approximately at any point at or near the rib, and the incision(s) preferably will be from about 2 to about 4 cm long, more preferably about 3 cm long. In recovering a phalanx, it is desirable to make incisions approximately at a proximal end of a proximal phalanx and/or a distal end of an associated metacarpal or metatarsal.

The recovery of any particular bone may alternatively be accomplished through a single strategic incision, without the need for additional incisions. A single incision recovery will generally be more difficult and complicated than a two incision recovery, but will also serve to further minimize the aesthetic detriment of the procedure.

The length of the incision will depend on its location. A hip incision will desirably be at least about 10 cm long and at most about 40 cm long, most preferably about 25 cm long, based on a donor having the size of an average adult male. A knee incision will desirably be at least about 6 cm long and at most about 50 cm long, most preferably about 20 cm long, based on a donor having the size of an average adult male. An ankle incision will desirably be at least about 6 cm long and at most about 40 cm long, most preferably about 15 cm long, based on a donor having the size of an average adult male. A shoulder incision will desirably be at least about 6 cm long and at most about 40 cm long, most preferably about 15 cm long, based on a donor having the size of an average adult male. An elbow incision will desirably be at least about 3 cm long and at most about 40 cm long, most preferably about 10 cm long, based on a donor having the size of an average adult male. A wrist incision will desirably be at least about 1 cm long and at most about 15 cm long, most preferably about 4 cm long, based on a donor having the size of an average adult male. For donors of sizes other than that of an average adult male, it will be desirable to adjust the desired incision lengths upward or downward, based on proportional size to an average adult male.

It has been found that a relatively small incision may be employed with the minimally invasive techniques described herein. For example, it is preferred that the incision is no more than about 40 cm long. Alternatively, the length of the incision can be limited to a proportion of the length of the underlying bone, such that the length is no more than about 50%, alternatively no more than about 33%, alternatively no more than about 25% of the length of the underlying bone.

The present techniques include methods and apparatus for separating soft tissue from bone or from other soft tissue. In recovering bone from a donor, the bone is generally separated from soft tissue, and it has been found desirable to leave some or all of the soft tissue in the donor as a way of maintaining the aesthetic appearance of the donor. The soft tissue may be separated without cutting the bone tissue, for example, by separating the tissue with a rounded instrument, such as a scissors.

The present techniques may also be used to recover groups of tissues as a unit, including multiple bones and tendons and ligaments connecting those bones. For example, a “leg en bloc” or “knee en bloc” may be recovered using the present techniques.

It is possible to use existing apparatus to perform the foregoing methods so long as the apparatus is suitable to tunnel along the bone. It is desirable to use a rounded, semi-sharp device for effective separation. The rounded edge of surgical scissors is an acceptable tool to perform the separation. This requires the recovery agent to manipulate the scissors both along the perimeter of a bone and along the length of the bone concurrently.

While it is possible to use existing apparatus for performing minimally invasive bone recovery methods, novel instruments are also provided herein for minimally invasive bone recovery methods and for separating soft tissue.

Instruments are provided for separating a bone from soft tissue surrounding the bone in a minimally invasive fashion. The instrument may comprise (a) an elongated shaft having a length and two ends; and (b) a tissue separator attached to one of the ends of the shaft. The tissue separator preferably has a moderately sharp edge, and the edge is essentially perpendicular to the length of the elongated shaft. A moderately sharp edge is sufficiently sharp to separate tissue from bone but insufficiently sharp to scrape or gouge the bone. The condition of the edge is crucial to preserving the integrity of vessels, vasculature, and other soft tissue surrounding the bone, and also crucial to the preservation of potentially implantable structures such as Achilles tendons, patellar tendons, and meniscus. As much as 25% of recovered tissue may be lost during processing due to damage caused by conventional recovery techniques. A moderately sharp edge reduces losses during graft processing by allowing separation of soft tissue from the bone with minimal damage to both the soft tissue and the bone. An edge which is too sharp can cut, gouge, scrape, or slice into either bone or soft tissue. An edge which is too blunt or dull will require excessive force, leading to tearing, stretching and pulling of the tissue. A moderately sharp edge is less sharp than a standard razor or scalpel. A moderately sharp edge is sufficiently sharp to separate the soft tissue from bone, yet not so sharp as to easily gouge into the bone itself. A moderately sharp edge is sharp enough to separate different tissues but not so sharp as to inadvertently transect intact tissue. For example, a moderately sharp edge is sufficiently sharp to separate a muscle from a tendon without cutting the tendon itself. The moderately sharp edge can be a lead edge, and the separator may also comprise a sharp edge on the back side.

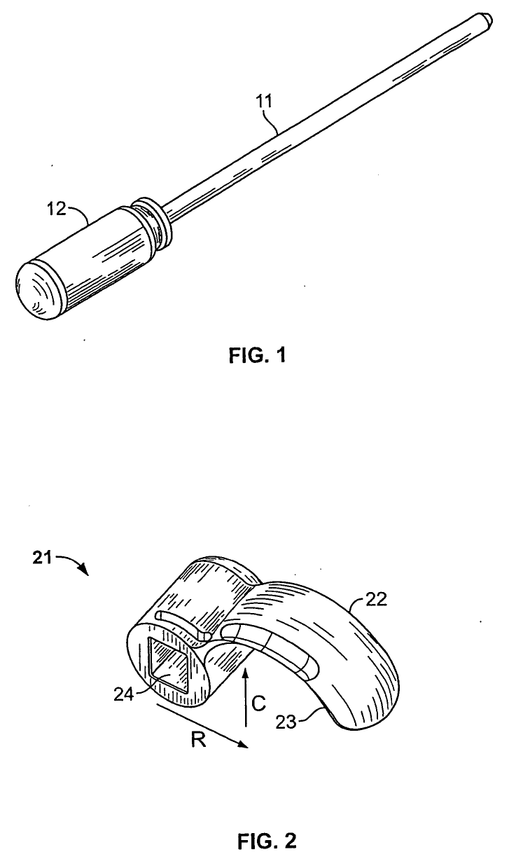

The shaft assembly can be at least about 6 cm long, alternatively at least about 10 cm long, alternatively at least about 20 cm long, alternatively at least about 30 cm long, alternatively at least about 40 cm long, alternatively at least about 50 cm long. The shaft assembly can include a handle at the end opposite to the separator. FIG. 1 shows shaft assembly comprising a shaft 11 and a handle 12. The edge can be offset from the shaft, for example by at least about 0.2 cm, alternatively at least about 0.5 cm, alternatively at least about 1 cm, alternatively at least about 2 cm.

Preferably the edge can have a shape that is reciprocal to at least a portion of the shape of a bone. For example, the edge can have a shape that is reciprocal to at least a portion of the exterior of a femur, a tibia, a fibula, a humerus, a radius, an ulna, an iliac crest, a pelvis, or another bone.

Alternatively, the separator (or an edge thereof) can have a circular shape. In such embodiments, the circular shape defines a radius, and the radius is from about 0.5 cm to about 20 cm. Alternatively, the separator (or an edge thereof) can have a U-shape. In such embodiments, the U-shape comprises two essentially parallel arms separated by a distance, and the distance is from about 0.5 cm to about 3.5 cm. Alternatively, the separator (or an edge thereof) can have a V-shape. In such embodiments, the V-shape comprises two arms separated by an angle and a corner radius. The included angle is from about o degrees to about 160 degrees, and the offset radius is from about 0 cm for a sharp point, to about 4 cm for a large round.

Alternatively, the separator (or an edge thereof) can have a suitable general shape, such as a semi-circular shape. A semi-circular shape is one having an essentially constant radius. In such embodiments, the semi-circular shape defines a radius, and the radius may be from about 1 cm to about 15 cm. Alternatively, the separator (or an edge thereof) can have a C-shape. A C-shape has variable or multiple radii and does not follow a single circle path. The C-shape starts on the circle, but leaves the path, either toward the inside or outside of the curve, near the end(s) of the C. In such embodiments, the C-shaped edge comprises two arms with endpoints separated by a distance of about 0.1 cm, alternatively about 0.5 cm, alternatively about 1 cm, alternatively about 2 cm, alternatively about 3 cm, alternatively about 5 cm, alternatively about 6 cm, alternatively about 8.5 cm. FIG. 2 shows a C-shaped separator 21 that is well suited for separating a femur from surrounding tissue. The separator 21 is C-shaped with rounded edges 22 and 23 on the leading and trailing edges to separate tissue from the femur as it slides along the length of the bone. It is contemplated the separator 21 defines an square opening 24 that receiving an end of a shaft (and preferably fits snugly with a square shaft end), such as the shaft 11 shown in FIG. 1. The separator is offset from the shaft axis by a distance R, which is preferably from about 0 cm to about 4 cm. The size of the separator may be selected according to the size of the donor bone, with an inner radius, C, that approximates the outer surface radius of the femur or other bone on which it is to be used. Preferred inner radii are from about 0.5 cm to about 10 cm. A kit comprising a shaft and several separators 21 of varying shapes and sizes may be provided. Alternatively, the kit may further comprise one or more separate handles attachable to one end of the shaft or directly to the tissue separators.

Alternatively, the separator (or an edge thereof) can have a J-shape. In such embodiments, the J-shape defines an angle, and preferably the angle is from about 30 degrees to about 160 degrees. FIG. 3 shows a J-shaped separator 31 that is well suited for separating a tibia from surrounding tissue. The J-shaped separator 31 has rounded edges 32 and 33 on the leading and trailing edges to separate tissue from the bone as it slides along the length of the bone. The J-shaped separator 31 shown in FIG. 3 has a shorter arm 35 farther from the shaft and a longer arm 36 closer to the shaft, but it is also contemplated that the arms can be the same size or that the shorter arm may be closer to the shaft and the longer arm farther from the shaft. The separator is sized with the “J” having an angle of A^{o}, which is preferably from about 60 degrees to about 160 degrees. The offset of the separator (the longer arm) from the shaft is a distance R, and the hook (the shorter arm) has a length J.

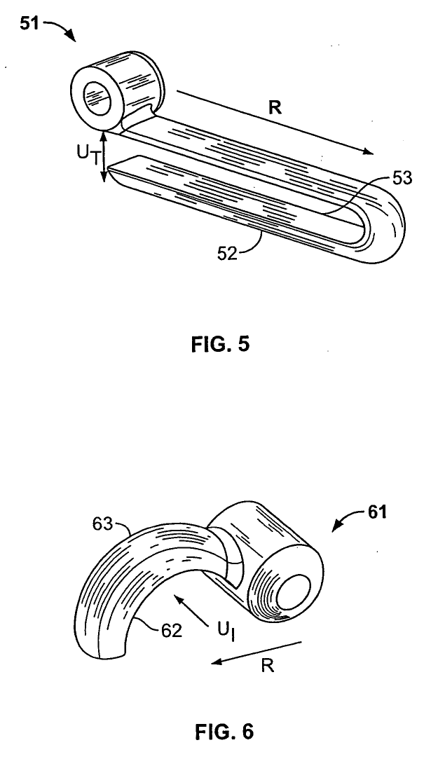

Alternatively, the separator (or an edge thereof) can form a closed loop, as exemplified by an O-shape. An O-shaped separator comprises an essentially circular edge, and a diameter in the range of from about 1 cm to about 15 cm. FIG. 4 shows an O-shaped separator 41 that is well suited for separating a fibula from surrounding tissue. Separator 41 is circular with rounded edges 42 and 43 on the leading and trailing edges to separate tissue from the bone as it slides along the length of the bone. The circular section is offset from the shaft by a distance R, and has a diameter of O. Alternatively, a closed-loop may contemplate irregular shapes which are generally round and also may contain one or more sharp edges such as a “D” shape, an oval, an ellipse, a conic section, a parabolic curve, or any closed loop shape.

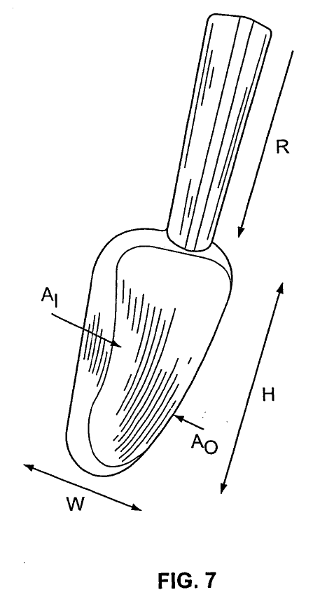

FIG. 5 shows a U-shaped separator that is well suited for separating a tendon or a portion of a tendon from surrounding tissue. The tendon separator tool shown in FIG. 5 is U-shaped with rounded edges on the leading and trailing edges to separate tissue from the tendon as it slides along the length of the tendon. The tool eases the separation of muscle and tissue from a tendon. The tool is configured with one end of the “U” being approximately tangent to the circle that makes the handle attachment and has a length R, which is preferably from about 0 cm to about 4 cm. The gap is oversized for the recovered tendon, and measures U_{T}. U_{T} is preferably from about 0.1 cm to about 2.5 cm.

FIG. 6 shows a U-shaped separator 61 that is well suited for separating an ilium from surrounding tissue. This U-shaped separator 61 has rounded edges 62 and 63 on the leading and trailing edges to separate tissue from the ilium as it slides along the length of the bone. The ends of the U shape may be rounded, blunted, skirted or flared out to prevent damage to the bone. The tool is sized with an inner radius, U_{I}, and is offset from the shaft by a radius R, which is preferably from about 0 cm to about 4 cm. U_{I} is preferably from about 0.1 cm to about 3.5 cm.

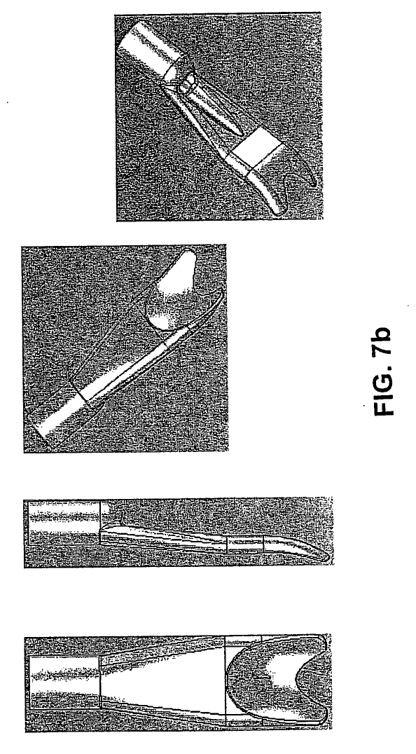

FIGS. 7(a) and 7(b) shows separators that are well suited for separating an Achilles tendon from surrounding tissue. The inside and outside surfaces of the scoop each has a single trailing edge to provide the separation between the tendon and the fatty tissue of the heel. The inner and outer radii of the scoop are A1 and AO. A1 is preferably from about 1 cm to about 5 cm, and AO is preferably from about 1 cm to about 10 cm. The scoop has a width W and a height H. The width W is preferably from about 0.5 cm to about 10 cm, and the height H is preferably from about 0.1 cm to about 15 cm. The scoop attaches to the shaft at a distance R from the top surface of the scoop. The distance R is preferably from about 0 cm to about 4 cm.

Although various shapes of separators have been described as being well suited for particular types of bones, it is contemplated that those shapes are also suitable for other types of bones as well, and nothing herein should be construed as indicating that a separator having a particular shape is limited to any particular bone.

The separator can be attached to the shaft in a permanent or removable manner. Preferably the separator is removably attached to the shaft by a suitable means for attachment. For example, the separator can be attached to the shaft by a screw-tapped surface, by a pin that intersects the shaft and the separator, by a screw that intersects the shaft and the separator, by a cotter pin, by a spring-loaded ball, or by a key that allows slip and twist. Preferably the separator is attached to the shaft by a threaded collar, as shown in FIG. 9.

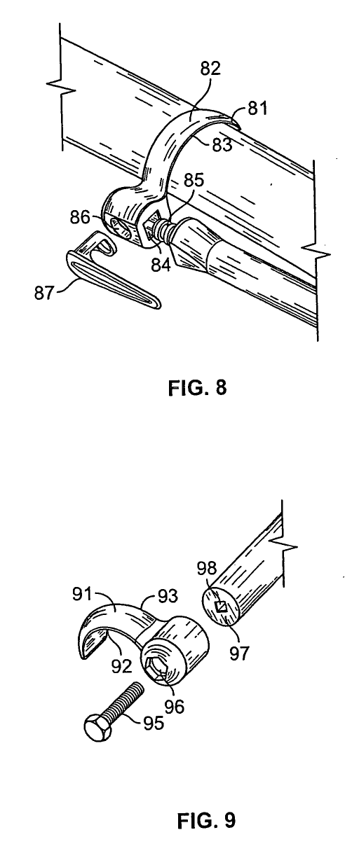

FIG. 8 shows a separator 81 attached to a shaft 85 with a cam arrangement. The separator 81 has a leading edge 82 and a trailing edge 83. The separator 81 has an opening 86 to accommodate a cam 87. The end of the shaft 85 is inserted in a reciprocal opening 84 in the separator, and the cam 87 is inserted in opening 86 and turn to hold the separator 81 on the shaft 85.

FIG. 9 shows a separator 91 attached to a shaft with a bolt 95. The separator 91 has a leading edge 92 and a trailing edge 93. The separator 91 is attached to the shaft by a bolt 95 whose head sits in a reciprocal recess 96 on the separator 91. The bolt 95 may end into an opening 98 on the shaft. The bolt 95 may be threaded so that it firmly attaches the separator 91 to the shaft. The end of the shaft has threads or teeth 97, and a bottom surface or recessed interior surface of the separator 91 has reciprocal threads or teeth which engage the threads or teeth 97 of the shaft to prevent the separator 91 from rotating around the shaft.

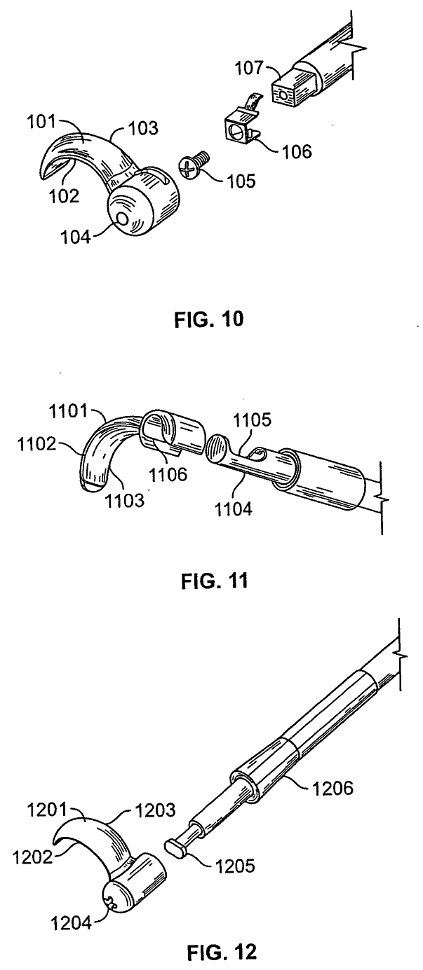

FIG. 10 shows a separator attached to a shaft with a leaf spring arrangement. The separator 1001 has a leading edge 1002 and a trailing edge 1003. A screw 1005 holds a leaf spring 1006 to the shaft, and the separator 1001 has an opening at its base that is reciprocal to the shape of the leaf spring, so that the separator can snap onto and be securely held by the leaf spring 1006. An opening 1004 provides access for a screwdriver to tighten or loosing the screw 1005.

FIG. 11 shows a separator attached to a shaft with a cut-out configuration. The separator 1101 has a leading edge 1102 and a trailing edge 1103. The shaft 1104 includes a cut-out 1105 which is reciprocal to a cut-out 1106 provided in the separator 1101. The mating of cut-outs 1105 and 1106 prevents the separator from rotating during use. A threaded sleeve 1107 is screwed against the bottom of the separator 1101 to securely but removably attach it to the shaft.

FIG. 12 shows a separator attached to a shaft with a T-shaped attachment. The separator 1201 has a leading edge 1202 and a trailing edge 1203. The shaft has a T-shaped tip 1205 which fits securely in an T-shaped or X-shaped opening 1204 defined by the top of the separator 1201. The secure fit of the T-shaped tip 1205 in the opening 1205 prevents rotation of the separator during use. A threaded sleeve 1206 is screwed against the bottom of the separator 1201 to securely but removably attach it to the shaft.

As another aspect of the present invention, instruments are provided for separating two soft tissues from one another in a minimally invasive fashion. The instrument comprises an elongated shaft having a length and two ends and a rigid tissue separator attached to one of the ends of the shaft. The tissue separator has a moderately sharp edge, the edge being essentially perpendicular to the length of the elongated shaft. The edge may have a U-shape or other suitable shape as described above. The separator can comprise an opening such that the separator is not a closed loop.

As yet another aspect, instruments are provided for separating an Achilles tendon from a donor in a minimally invasive fashion. The instrument comprises an elongated shaft having a length and two ends and a tissue separator attached at one end of the shaft, wherein the separator comprises a scoop. The scoop can be oriented along the length of the shaft. The scoop can have has inside and outside surfaces, and each surface has a single trailing edge to provide separation between the tendon and the skin by dividing the fatty tissue of the heel. The scoop can have an inner radius of from about 0.5 cm to about 5 cm and an outer radius of from about 0.1 cm to about 5 cm. Alternatively, the scoop can be flat or nearly flat with either inner or outer radius greater than about 5 cm to an infinite effective radius for a flat scoop. The scoop can have a height of from about 1 cm to about 30 cm and a width of from about 0.1 cm to about 10 cm.

Though the present techniques have generally been described in the context of recovering bone with reduced detriment to the aesthetic appearance of the donor, it has also been recognized that the present techniques may be used to recover other types of tissue. For example, as already described in connection with FIG. 7, an Achilles tendon may be recovered in a minimally invasive manner from a living or deceased donor. More broadly, the present techniques provided methods and materials for recovering tendon grafts in a minimally invasive fashion. The methods comprise separating the tendon from an associated muscle; removing the tendon; and leaving most of the associated muscle essentially intact in the donor's body. By way of example, the following tendons may be separated and recovered from the associated muscles of the same name: biceps femoris, peroneus brevis, peroneus tertius, peroneus longus, sartorius, anterior tibialis, posterior tibialis, plantaris, extensor hallucis longus, flexor digitorum longus, extensor digitorum longus, superior extensor retinaculum, interior extensor retinaculum, semitendinosus, semibranosus or gracilis. Additionally, the quadriceps tendon may be separated and recovered from the rectus femoris, vastus lateralis, vastus medialis, and vastus intermedius muscles; the Achilles tendon may be separated and recovered from the soleus and gastrocnemius muscles; and the tensor fascia latae may be separated and recovered.

As a further aspect, kits are provided for procuring an essentially intact bone from a donor with reduced detriment to the aesthetic appearance of the donor. One such kit comprises an elongated shaft having a length and two ends and two or more tissue separators. The tissue separators may have substantially similar shapes but have different sizes (different lengths, radii, and/or angles), and/or may be different shapes. Each of the tissue separators in the kit preferably is selected from the group consisting of: separators comprising a separator edge having a semi-circular shape; separators comprising a separator edge having a circular shape; separators comprising a separator edge having a J-shape; separators comprising a separator edge having a C-shape; separators comprising a separator edge having a U-shape; and separators comprising a separator edge having a scoop shape; wherein each of the separators is attachable to one of the ends of the shaft. Alternatively, multiple shafts of varying lengths, diameters, cross sections, or shapes may be provided in a kit.

Other tool kits are expected to be facilitate the minimally invasive techniques. For example, a kit containing six (6) separators and a single handle is contemplated. The shaft design is such that the devices can easily be attached and removed by the recovery agent during a recovery. Optionally, the entire kit can be re-used following decontamination and or sterilization. The interchangeable handle shown in FIG. 1 attaches to all of the devices simply. The attachment method is such that sterilization of the handle and separators is easily obtained. Alternatively, one or more of the kit components may be a single use or disposable design.

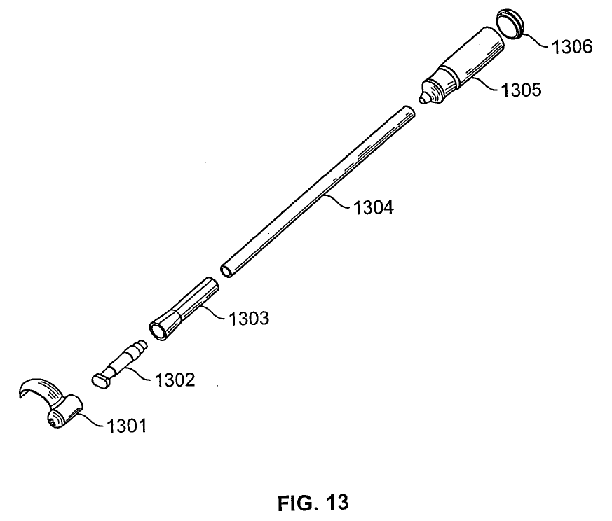

For further illustration of the present instruments, FIG. 13 shows an exploded view of one embodiment of an instrument for separating bone from surrounding tissue. FIG. 13 shows a separator 1301, a T-shaped tip 1302, a threaded sleeve 1303, a shaft 1304, a handle 1305, and a handle cap 1306. Of course additional parts may be included in the instrument for various purposes.

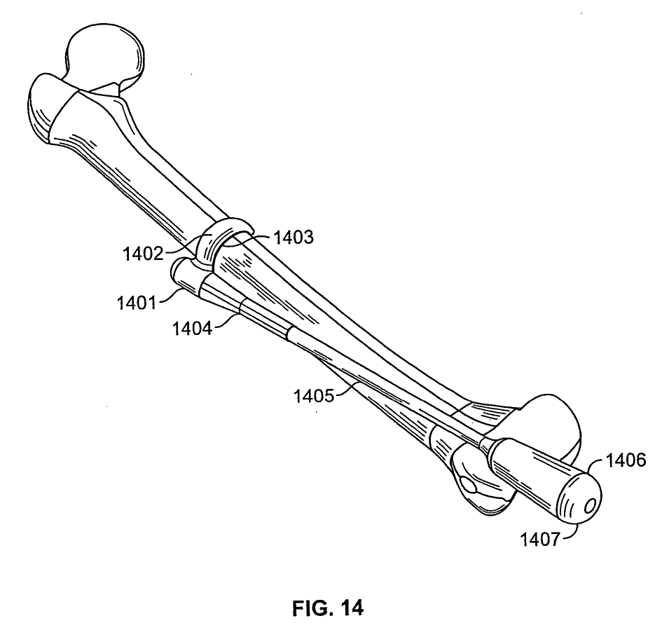

FIG. 14 depicts the use of one embodiment of an instrument for separating bone from surrounding tissue. In this depiction, the interaction of an instrument with a long bone (in this case, a femur) is shown. For clarity in showing this interaction, the surrounding tissue is not shown, but in actual use, the separator would separate surrounding tissue from the bone as the separator moved along the bone. The instrument includes a separator 1401 having a leading edge 1402 and a trailing edge 1403, one or both of which may be sharp or moderately sharp edges. The separator 1401 is attached by a threaded collar 1404 to a shaft 1405. For the convenience of the recovery agent, a handle 1406 is attached to the shaft 1405 and is held on by a handle cap 1407.

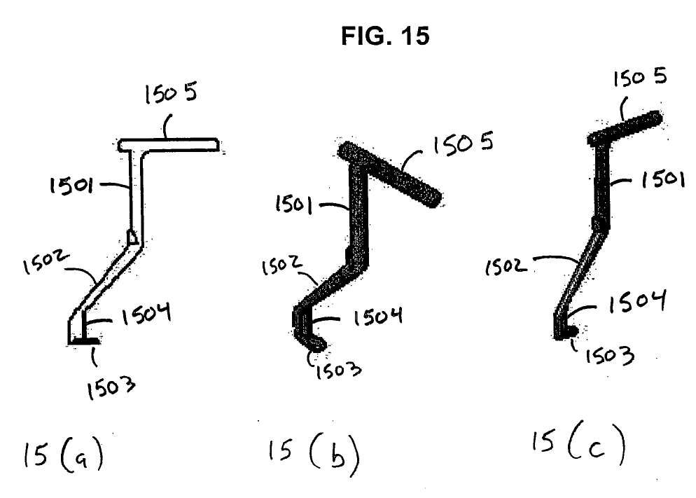

FIG. 15 shows three views (15(a), 15(b) and 15(c)) of an instrument comprising a sternal knife. The sternal knife differs from current tools (for example, the Lebsche tool) by having an offset striking plate 1501 and a changed shape of the blade guide 1502. The instrument features a larger guide, in the shape of a rounded wing 1503, to keep the knife from slipping out of the sternum cavity while forcefully cutting. The separator includes a sternum knife 1504 for cutting. The offset keeps the driving mallet or hammer away from the patients' face and provides a visual line of sight to verify position of tip while cutting. The sternal instrument preferably from about 12 cm to about 40 cm from the tip of blade to the tip of handle, and from about 5 cm to about 40 cm long in the direction of cutting, and the separator wings 1503 may be 1 cm to 5 cm wide from tip to tip. The wings lift the sternum up while holding the instrument beneath sternum. This makes it easier to avoid damaging the heart and other internal organs and prevents accidental slippage and release of the instrument from the sternum. The handle 1505 can also be offset to either side of the cut to facilitate cutting, alignment, and visual feedback.

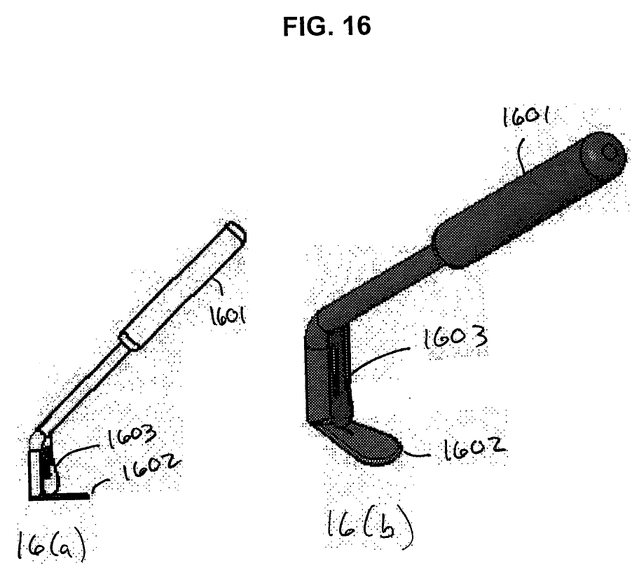

FIG. 16 shows two views (16(a) and 16(b)) of a knife well suited for separating skin or fascia. The instrument comprises a handle 1601 and features a rounded wing 1602 (similar to the sternal instrument). The wing 1602 is significantly thinner than the sternal separator, to assist the cutting of skin and separation from surrounding tissue. The wing separates the skin from internal soft tissue, so the blade 1603 will only cut the skin or cut just below skin, but not into the muscle or connective tissue below. The blade may be a standard “No. 20” surgical steel blade. The wing 1602 lifts the skin, holds the blade 1603 below the skin to avoid unintentional lift out, and helps to separate skin from surrounding tissue. The handle preferably is between 6 cm and 20 cm in length, the wingspan preferably is about 0 to 3 cm wide, and the offset angle between the handle and the plane of cutting under the skin preferably is between 30 degrees where the handle would lead or pull the wing and 150 degrees where the handle would trail or push the wing. The handle can also be offset to either side of the cut to facilitate cutting, alignment, and visual feedback.

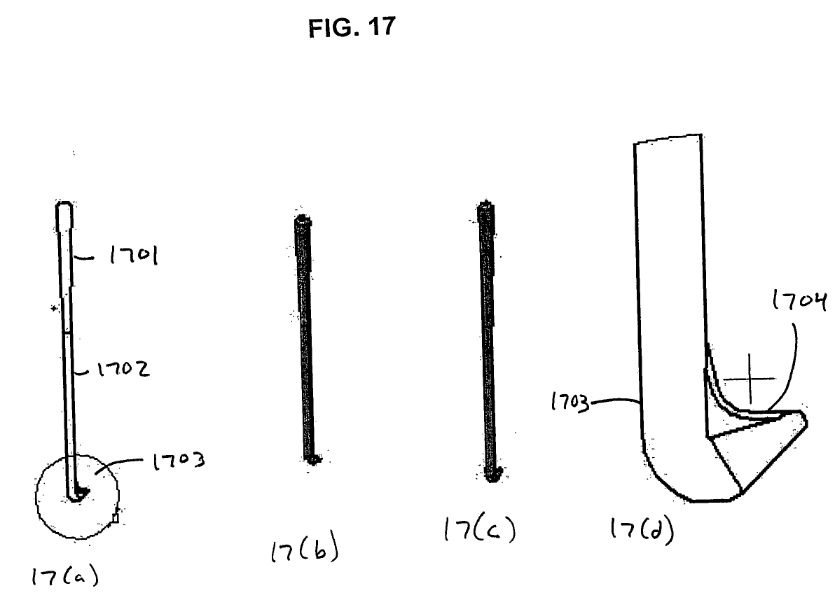

FIG. 17 shows a curved knife well suited for dissecting and/or transecting tendon, ligament, retinaculum, and/or other soft tissue. The instrument comprises a handle 1701, a shaft 1702, and a separator 1703. The instrument allows the user to easily avoid bones and meniscus when cutting tendons and ligaments in the region of the knee. This is accomplished with slender shafting and a concise hooked cutting surface 1704. The instrument can also be used to cut many other tissues by hooking and pulling.

### EXAMPLES

The following nonlimiting examples will further illustrate the invention.

### Example 1

The example demonstrates the method of using conventional instruments. Recovering bone and tissue from the lower extremity, the minimally invasive process used strategic incisions at the top of the hip, the knee, and the ankle, rather than the “Watson Jones” type incision that runs the entire length of the leg. The first strategic incision started above the posterior half of iliac crest that follows the sartorial tract to the level of the greater trochanter of the femur. The incision was approximately 25 cm in length. The strategic midline incision at the knee was approximately 20 cm in length with the patella being the midpoint. The strategic incision at the ankle was approximately 15 cm in length with anterior ankle being the midpoint of the incision.

After the incisions were made, an instrument comprising a moderately sharp edge (a surgical scissors) was inserted into the incision. The edge was contacted with the bone to be recovered at the interface of the bone and its surrounding tissue, and the edge was used to push the surrounding tissue away from the bone. The edge generally did not cut into the surrounding tissue and did not cut deeply into the surrounding tissue. This separation procedure was repeated at each of the incisions, so that all the surrounding tissue was separated from the bone to be removed. After the surrounding tissue was separated, the bone was removed from the donor through one of the incisions.

### Example 2

In this example, an instrument comprising a handle as shown in FIG. 1 and separators as shown in FIGS. 2, 3 and 4 is used for the recovery of the long bones from a deceased donor in a minimally invasive fashion. A human femur, tibia and fibula are recovered from a cadaver in the following manner. The cadaver is an adult male of average size. Strategic incisions are made as described in Example 1. The femur separator is securely attached to the shaft and the separator is inserted in the incision at the hip and used to separate surrounding tissue from the top portion of the femur. Next, the separator is inserted in the incision at the knee and used to separate surrounding tissue from the bottom portion of the femur.

The femur separator is then detached from the shaft, and the tibia separator is attached to the shaft. The separator is inserted in the incision at the knee and used to separate surrounding tissue from the top portion of the tibia. The separator is inserted in the incision at the ankle and used to separate surrounding tissue from the bottom portion of the tibia. Next these steps are repeated on the fibula using the fibula separator. After the surrounding tissues are separated, the femur, tibia and fibula are removed from the donor's leg. The incisions are then sutured. Because there has been minimal damage to the vascular systems of the surrounding tissues, an embalming fluid may be passed into those vascular systems for preserving the donor's body for burial.

### Example 3

The patient is prepped and draped and then the acromion and antecubital fossa are identified. A midline skin deep incision is made with a scalpel in-line with the biceps muscle from the acromion to approximately 8 cm distally. A midline incision at the antecubital fossa is made approximately 6 cm, where two thirds of the incision lies on the humeral side. A second scalpel is used to extend both incisions through the underlying muscle to the level of the humerus.

The soft tissue surrounding the diaphysis of the humerus is separated using a musculotendinous separator. The humeral head is separated from its attachments to the acromion and glenoid fossa with sharp and blunt dissection techniques. The humeral condyles and distal humerous are then freed from their attachments to the radius and ulna using sharp and blunt dissection. Finally, the humerus is removed by either grasping the humeral head and acromial incision or grasping the humeral condyles and antecubital fossa incision.

Once removed, the humerus is transferred to the back table to be packaged. A prosthesis is then selected and placed in the humeral bed, and the patients' incisions are closed.

All patents, test procedures, and other documents cited herein, including priority documents, are fully incorporated by reference to the extent such disclosure is not inconsistent with this invention and for all jurisdictions in which such incorporation is permitted.

While the present invention has been described and illustrated by reference to particular embodiments, it will be appreciated by those of ordinary skill in the art that the invention lends itself to many different variations not illustrated herein. For these reasons, then, reference should be made solely to the appended claims for purposes of determining the true scope of the present invention.

Although the appendant claims have single appendencies in accordance with U.S. patent practice, each of the features in any of the appendant claims can be combined with each of the features of other appendant claims or the main claim.

What is claimed is:

1. A method for minimally invasive recovery of tissue from a donor, the method comprising:
accessing an essentially intact tissue through a strategic incision;
removing the essentially intact tissue from the donor; and
leaving the surrounding tissue essentially intact inside the donor.

2. The method of claim 1, wherein the surrounding tissue comprises a vascular network, and the method comprises separating the surrounding tissue from the bone, while leaving essentially intact the vascular network of the surrounding tissue.

3. The method of claim 1, further comprising the step of passing a fluid through the vascular network of the surrounding tissue.

4. The method of claim 1, where the fluid is an embalming fluid.

5. A method for recovering a substantial portion of a bone from a donor with reduced detriment to the aesthetic appearance of the donor, the bone having a proximal third, a distal third, and a central third, the method comprising:
(a) making an incision within the proximal or distal third of the bone;
(b) separating soft tissue surrounding the bone along at least a portion of the length of the bone by accessing the bone and the tissue through the incision; and
(c) removing a substantial portion of the bone through the incision.

6. The method of claim 5 wherein the substantial portion of the bone is essentially intact bone.

7. The method of claim 5 wherein the incision is made at the articulation of two connected bones.

8. The method of claim 5 leaving essentially all of the surrounding tissue in the donor.

9. The method of claim 5 wherein the donor is a cadaver.

10. The method of claim 5 wherein the donor is a living patient.

11. A method for removing an essentially intact tissue with reduced detriment to the donor's body, the method comprising:
accessing an essentially intact tissue through an opening having a length of not more than about 50 cm,
separating the essentially intact tissue from surrounding tissue,
removing the essentially intact tissue from the donor's body, and
leaving the surrounding tissue essentially intact inside the donor's body.

12. The method of claim 11, wherein the opening is an incision through the donor's skin.

13. The method of claim 11, wherein the surrounding tissue comprises a vascular network, and the method comprises separating the surrounding tissue from the bone, while leaving essentially intact the vascular network of the surrounding tissue.

14. The method of claim 11, further comprising the step of passing a fluid through the vascular network of the surrounding tissue.

15. The method of claim 11, where the fluid is an embalming fluid

16. A method for separating a bone from surrounding tissue in a minimally invasive fashion, the bone having a length and two ends, the method comprising:
(a) making an incision which does not pass through the central third of the length of the bone; and
(b) accessing the bone through the incision; and
(c) separating the surrounding tissue from the bone along a majority of the perimeter and at least a portion of the length of the bone.

17. The method of claim 16 wherein the incision does not extend into the central third of the length of the bone.

18. The method of claim 16 wherein the incision passes through articulation of two connected bones.

19. The method of claim 16 wherein the incision has a midpoint about at the center of the articulation.

20. The method of claim 16 further comprising making a second incision at the other end of the bone and separating soft tissue along the circumference at least a second portion of the length of the bone through the second incision.

21. The method of claim 16 wherein the bone is disposed in an extremity, and the method comprises making no more than three incisions in the extremity.

22. The method of claim 16 wherein the incision is no more than about 20 cm long in the upper extremity.

23. The method of claim 16 wherein the incision is no more than about 30 cm long in the lower extremity.

24. The method of claim 16 wherein the length of the incision is no more than about 33% of the length of the bone.

25. The method of claim 16 wherein the incision is located no more than about 20 cm from the end of the bone in the upper extremity.

26. The method of claim 16 wherein the incision is located no more than about 30 cm from the end of the bone in the lower extremity.

27. The method of claim 16 wherein the incision is approximately at the hip and is about 2 cm above the tissue at or near the anterior portion of an iliac crest.

28. The method of claim 16 wherein the bone is a femur, and the incisions are approximately at a hip and a knee.

29. The method of claim 16 wherein the bone is a tibia, and the incisions are approximately at a knee and an ankle.

30. The method of claim 16 wherein the bone is a fibula, and the incisions are approximately at a knee and an ankle.

31. The method of claim 16 wherein the incision is approximately at the hip and is less than about 40 cm long.

32. The method of claim 16 wherein the incision is approximately at the knee and is less than about 35 cm long.

33. The method of claim 16 wherein the incision is approximately at the ankle and is less than about 30 cm long.

34. The method of claim 16 wherein the incision is approximately at the hip and is about 25 cm long.

35. The method of claim 16 wherein the incision is approximately at the knee and is about 20 cm long.

36. The method of claim 16 wherein the incision is approximately at the ankle and is about 15 cm long.

37. The method of claim 16 wherein the bone is a humerus, and the incisions are approximately at a shoulder and an elbow.

38. The method of claim 16 wherein the bone is a radius, and the incisions are approximately at an elbow and a wrist.

39. The method of claim 16 wherein the bone is an ulna, and the incisions are approximately at an elbow and a wrist.

40. The method of claim 16 wherein the bone is a scapula, and the incision is approximately at a shoulder.

41. The method of claim 16 wherein the bone is a rib, and the incisions are approximately at any point about the rib and from about 2 to about 4 cm long.

42. The method of claim 16 wherein the bone is a phalanx, and the incisions are approximately at a proximal end of a proximal phalanx or a distal end of an associated metacarpal or metatarsal.

43. The method of claim 16 wherein the incision is approximately at the shoulder and is less than about 30 cm long.

44. The method of claim 16 wherein the incision is approximately at the elbow and is about 25 cm long.

45. The method of claim 16 wherein the incision is approximately at the shoulder and is about 15 cm long.

46. The method of claim 16 wherein the incision is approximately at the elbow and is about 10 cm long.

47. The method of claim 16 wherein the incision is approximately at the wrist and is about 10 cm long.

48. The method of claim 16 wherein the incision is approximately at the wrist and is about 4 cm long.

49. The method of claim 16 separating the soft tissue from bone without dividing the bone tissue.

50. The method of claim 16 further comprising the step of dividing the bone into pieces to facilitate removal.

51. The method of claim 16 separating the tissue with a rounded instrument.

52. The method of claim 16 separating the tissue with scissors.

53. A method for removing a long bone or a substantial portion of a long bone from a limb of a deceased donor, with reduced detriment to the appearance of the donor's body, the method comprising:
accessing the long bone through a strategic incision which spans less than the length of the long bone;
separating the long bone from the surrounding tissue; and
removing the long bone or substantial portion of the long bone from the limb.

54. The method of claim 53, wherein the surrounding tissue is left essentially intact inside the limb.

55. The method of claim 53, wherein the long bone is removed essentially intact from the limb.

56. The method of claim 53, further comprising replacing the long bone with a prosthesis which restores the appearance of the donor's body.

57. The method of claim 53, further comprising closing the strategic incisions to restore the appearance of the donor's body.

58. The method of claim 53 wherein the strategic incision has a length that is less than ⅔ of the length of the long bone.

59. The method of claim 53 wherein the strategic incision has a length that is less than ½ of the length of the long bone.

60. The method of claim 53 wherein the strategic incision has a length that is less than ⅓ of the length of the long bone.

61. The method of claim 53 wherein the strategic incision has a length that is less than ¼ of the length of the long bone.

62. The method of claim 53 wherein the strategic incision has a length that is less than ⅕ of the length of the long bone.

63. The method of claim 53 wherein the strategic incision has a length that is less than 1/10 of the length of the long bone.

64. A method for recovering an essentially intact bone or substantial portion of a bone from a donor with reduced detriment to the appearance of the donor, the method comprising:
a) separating the bone from essentially all surrounding soft tissue;
b) removing the bone or a substantial portion of the bone from the donor's body; and
c) leaving the soft tissue essentially intact in the donor's body

65. A method for recovering an essentially intact bone or substantial portion of a bone from a donor with reduced detriment to the appearance of the donor, the method comprising:
(a) accessing the long bone through a strategic incision having a length which is less than the length of the long bone;
(b) removing the bone or a substantial portion of bone from the donor's body; and
(c) leaving the soft tissue essentially intact in the donor's body.

66. The method of claim 65 wherein the strategic incision has a length that is less than ⅔ of the length of the long bone.

67. The method of claim 65 wherein the strategic incision has a length that is less than ½ of the length of the long bone.

68. The method of claim 65 wherein the strategic incision has a length that is less than ⅓ of the length of the long bone.

69. The method of claim 65 wherein the strategic incision has a length that is less than ¼ of the length of the long bone.

70. The method of claim 65 wherein the strategic incision has a length that is less than ⅕ of the length of the long bone.

71. A method for separating a bone from surrounding tissue with reduced detriment to the surrounding tissue, the method comprising:
(a) making an incision which provides access to the bone while leaving the surrounding tissue essentially intact;
(b) positioning a moderately sharp edge at a surface of the bone through the incision;
(c) moving the moderately sharp edge between the bone and the surrounding tissue for at least part of the length of the bone.

72. A method for removing a tendon from a limb of a deceased donor, the limb having a length, the method comprising:
accessing the tendon through a strategic incision having a length less than twice the length of the tendon;
separating the tendon from surrounding tissue; and
removing the tendon from the limb.

73. The method of claim 72, wherein the surrounding tissue is left essentially intact inside the limb.

74. The method of claim 72, wherein the tendon removed from the limb is essentially intact.

75. The method of claim 72, wherein the tendon is attached to one or more portions of bone, and the attached bone portions are removed from the limb with the tendon.

76. The method of claim 72, further comprising closing the strategic incisions to restore the appearance of the donor's body.

77. An instrument for separating a bone from soft tissue surrounding the bone in a minimally invasive fashion, the instrument comprising:
(a) an elongated shaft having a length and two ends; and
(b) a tissue separator attached to one of the ends of the shaft, the tissue separator having a moderately sharp edge, the edge being essentially perpendicular to the length of the elongated shaft.

78. The instrument of claim 77, wherein the shaft comprises a plurality of connected segments.

79. The instrument of claim 77, wherein each of the segments are removably connected.

80. The instrument of claim 77, wherein the shaft comprises a plurality of telescoping segments.

81. The instrument of claim 77, further comprising a handle attached to the shaft at the end of the shaft opposite to the separator.

82. The instrument of claim 77 wherein the moderately sharp edge is a lead edge, and the separator further comprises a sharp edge on the back side.

83. The instrument of claim 77 wherein the shaft assembly is at least about 15 cm long.

84. The instrument of claim 77 wherein the shaft assembly comprises a handle at the end opposite to the separator.

85. The instrument of claim 77 wherein the edge is offset from the shaft by at least about 0.5 cm.

86. The instrument of claim 77 wherein the edge has a shape that is reciprocal to at least a portion of the shape of a bone.

87. The instrument of claim 77 wherein the edge has a shape that is reciprocal to at least a portion of the exterior of a femur.

88. The instrument of claim 77 wherein the edge has a shape that is reciprocal to at least a portion of the exterior of a tibia.

89. The instrument of claim 77 wherein the edge has a shape that is reciprocal to at least a portion of the exterior of a fibula.

90. The instrument of claim 77 wherein the edge has a shape that is reciprocal to at least a portion of the exterior of a humerus.

91. The instrument of claim 77 wherein the edge has a shape that is reciprocal to at least a portion of the exterior of a radius.

92. The instrument of claim 77 wherein the edge has a shape that is reciprocal to at least a portion of the exterior of an ulna.

93. The instrument of claim 77 wherein the edge has a shape that is reciprocal to at least a portion of the exterior of an iliac crest.

94. The instrument of claim 77 wherein the edge has a shape that is reciprocal to at least a portion of the exterior of a pelvis.

95. The instrument of claim 77 wherein the edge has a semi-circular shape.

96. The instrument of claim 77 wherein the semi-circular edge defines a radius, and the radius is from about 1 cm to about 15 cm.

97. The instrument of claim 77 wherein the edge has a circular shape.

98. The instrument of claim 77 wherein the circular edge defines a radius, and the radius is from about 0.5 cm to about 20 cm.

99. The instrument of claim 77 wherein the edge has a J-shape.

100. The instrument of claim 77 wherein the J-shaped edge defines an angle, and the angle is from about 60 degrees to about 160 degrees.

101. The instrument of claim 77 wherein the edge has a U-shape.

102. The instrument of claim 77 wherein the U-shaped edge comprises two essentially parallel arms separated by a distance, and the distance is from about 0.5 cm to about 3.5 cm.

103. The instrument of claim 77 wherein the edge has a C-shape.

104. The instrument of claim 77 wherein the C-shaped edge comprises two arms with endpoints separated by a distance, and the distance is from about 0.1 cm to about 8.5 cm.

105. The instrument of claim 77 wherein the edge has a O-shape.

106. The instrument of claim 77 wherein the O-shaped edge comprises an essentially circular edge, and the diameter is from about 0.5 cm to about 20 cm.

107. The instrument of claim 77 wherein the separator is removably attached to the shaft.

108. The instrument of claim 77 wherein the separator is attached to the shaft by a screw-tapped surface.

109. The instrument of claim 77 wherein the separator is attached to the shaft by a pin that intersects the shaft and the separator.

110. The instrument of claim 77 wherein the separator is attached to the shaft by a screw that intersects the shaft and the separator.

111. The instrument of claim 77 wherein the separator is attached to the shaft by a cotter pin.

112. The instrument of claim 77 wherein the separator is attached to the shaft by a spring-loaded ball.

113. The instrument of claim 77 wherein the separator is attached to the shaft by a key that allows slip and twist, and is locked in place with a threaded collar.

114. An instrument for separating two soft tissues from one another in a minimally invasive fashion, the instrument comprising:
an elongated shaft having a length and two ends; and
a rigid tissue separator attached to one of the ends of the shaft, the tissue separator having a moderately sharp edge, the edge being essentially perpendicular to the length of the elongated shaft.

115. The instrument of claim 114 wherein the edge has a U-shape.

116. The instrument of claim 114 wherein the separator comprises an opening such that the separator is not a closed loop.

117. An instrument for separating an Achilles tendon from a donor in a minimally invasive fashion, the instrument comprising:
an elongated shaft having a length and two ends;
a tissue separator attached at one end of the shaft, wherein the separator comprises a scoop.

118. The method of claim 117 wherein the scoop is oriented along the length of the shaft.

119. The method of claim 117 wherein the scoop has inside and outside surfaces, and each surface has a single trailing edge.

120. The method of claim 117 wherein the scoop has an inner radius of from about 1 cm to about 5 cm and an outer radius of from about 0.5 cm to about 10 cm.

121. The method of claim 117 wherein the scoop has a height of from about 0.1 cm to about 15 cm and a width of from about 0.5 cm to about 10 cm.

122. A kit for procuring an essentially intact bone from a donor with reduced detriment to the donor, the kit comprising:
an elongated shaft having a length and two ends;
two or more tissue separators, wherein each of the tissue separators is selected from the group consisting of:
separators comprising a separator edge having a semi-circular shape;
separators comprising a separator edge having a circular shape;
separators comprising a separator edge having a J-shape;
separators comprising a separator edge having a C-shape;
separators comprising a separator edge having a U-shape; and
separators comprising a separator edge having a scoop shape;
wherein each of the separators is attachable to one of the ends of the shaft.

123. A kit for procuring a bone from a donor with reduced detriment to the donor, the kit comprising:
an elongated shaft having a length and two ends;
two or more tissue separators, wherein each of the two or more tissue separators as a substantially similar shape but a different size than another of the separators.
